(19) United States
(12) Patent Application Publication
(10) Pub. No.: US 2011/0097710 A1
(43) Pub. Date: Apr. 28, 2011

(54) Methods for detecting atrial fibrillation and related conditions

(76) Inventors: Calum A. Macrae, Newton Center, MA (US); Patrick T. Ellinor, Brookline, MA (US); Adrian F. Low, Boston, MA (US)

(21) Appl. No.: 11/666,574

(22) PCT Filed: Oct. 25, 2005
(86) PCT No.: PCT/US05/38482

§ 371 Date: Feb. 27, 2009

Related U.S. Application Data
(60) Provisional application No. 60/622,155, filed on Oct. 26, 2004.

(51) Int. Cl. C12Q 1/68; G01N 33/68; G01N 33/53

(52) U.S. Cl. 435/6; 436/86; 436/501; 436/94; 435/29; 435/7.1

(57) ABSTRACT

Methods for detecting disorders associated with atrial fibrillation, and, thus, at risk for stroke and/or heart failure) in a subject based on a monitoring plasma levels of apelin are provided. Diagnostic compositions for the detection of such disorders are additionally provided.

### RELATED APPLICATIONS/PATENTS & INCORPORATION BY REFERENCE

This application claims priority to U.S. Provisional Application Ser. No. 60/622,155, filed on Oct. 26, 2004, the contents of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF GOVERNMENTAL INTEREST

This work was funded in part by grant number N1K23 from the National Institutes of Health. Accordingly, the United States Government may have certain rights to this invention.

### BACKGROUND OF THE INVENTION

Atrial fibrillation is the most common cardiac arrhythmia and is associated with significant morbidity and mortality (S S. Chugh et al. Am Coll Cardiol. 2001; 37:371-8). Atrial fibrillation is a disorder found in about 2.2 million Americans. In it, the two small upper chambers of the heart (the atria) quiver instead of beating effectively. Blood is not pumped completely out of the chambers, so it may pool and clot. If a piece of a blood clot in the atria leaves the heart and becomes lodged in an artery in the brain, a stroke results. About 15 percent of strokes occur in people with atrial fibrillation. The likelihood of developing atrial fibrillation increases with age. Three to five percent of people over 65 have atrial fibrillation.

Atrial fibrillation is also a major independent risk factor for the subsequent development of left ventricular dysfunction, with as many as 45% of those affected by the arrhythmia developing congestive heart failure within 10 years (T J. Wang et al. Circulation 2003; 107:2920-5). Dissection of the etiology of atrial fibrillation is likely to shed light not only on the mechanisms of this arrhythmia, but also may offer insights into the early pathobiology of congestive heart failure.

Recent work has identified a substantial heritable contribution to atrial fibrillation, raising the possibility of intrinsic differences in the individual threshold for this arrhythmia (D. Darbar et al. Journal of the American College of Cardiology 2003: 41:2185-92 and C S. Fox et al. Jama. 2004; 291-2851-5). Biomarkers could possibly identify those individuals with an increased propensity to atrial fibrillation. The ability to detect those with atrial fibrillation or at risk for developing it would aid in the clinical management of these individuals and related conditions such as stroke or congestive heart failure.

The APJ receptor belongs to a family of 7-transmembrane G-protein coupled receptors first cloned in 1993 (B F. O'Dowd et al. Gene 1993; 136:355-60). Orphaned for many years, its endogenous ligand, apelin, was subsequently isolated (K. Tatemoto et al. Biochem Piophys Res Commun. 1998; 251-471-6). Apelin is an endogenous peptide hormone that appears to have a physiologic role in counter-regulation of the angiotensin and vasopressin systems. This peptide has been reported to be downregulated in subjects with acute heart failure, but has not been studied in other cardiovascular conditions.

Expression of both the APJ receptor and its ligand parallel that of the angiotensin receptor AT1 and angiotensinogen respectively, suggesting a role in similar biological processes^{12,13}. Intraperitoneal administration of apelin in rats has been shown to result in short-term increase in drinking behaviour, a finding similar to the thirst-promoting effect of angiotensin II (D K Lee et al. J. Neurochem 2000; 73:34-41). However, administration of intravenous apelin lowered blood pressure in anesthetized rats through a nitric oxide dependent pathway (K. Tatemoto et al. Regul Pept. 2001; 99:87-92). Apelin also is one of the most potent endogenous positive inotropic substances yet identified (I. Szokodi et al. Circ. Res. 2002; 91:434-40). These results, combined with recent evidence of increased pressor responses in mice null at the APJ receptor, suggest that the apelin-APJ axis plays an important counter-regulatory role to the effects of angiotensin (J. Ishida et al. J. Biol. Chem. 2004:279-26274-9).

Circulating apelin levels are elevated early in the natural history of heart failure, but ultimately are depressed in overt congestive heart failure (G. Foldes et al. Biophys Res Commun 2003; 308:480-5). Following ventricular offloading in severe heart failure using a left ventricular assist device, expression of the APJ gene is markedly upregulated while the natriuretic peptide genes are downregulated (MM. Chen et al. Circulation 2003; 108:1432-9). Thus, the role of apelin in heart disorders has not been ascertained.

### SUMMARY OF THE INVENTION

In a homogeneous group of patients with lone atrial fibrillation (AF), a highly significant reduction of plasma apelin levels was found when compared to a matched control population. Accordingly, the use of the hormone apelin as a marker for the predisposition to atrial fibrillation and congestive heart failure is described herein.

In one aspect, the invention provides a method of detecting a disorder associated with a cardiac arrhythmia in a subject having said disorder comprising measuring the amount of an apelin polypeptide, or a fragment thereof, in the plasma or a preparation thereof of the subject, and determining that the amount is reduced in comparison to that of a control subject, thereby detecting said disorder. The measuring can comprise contacting the plasma or a preparation thereof with an immunological agent the binds with specificity to the apelin polypeptide or fragment thereof.

In another aspect, the invention provides a method of detecting a disorder associated with a cardiac arrhythmia in a subject having said disorder comprising measuring the amount of an apelin nucleotide sequence in circulating cells or in the plasma or a preparation thereof of the subject, and determining that the amount is reduced in comparison to that of a control subject, thereby detecting said disorder. The measuring can comprise contacting the circulating cells or plasma or a preparation thereof with at least one nucleic acid probe having a nucleic acid sequence complementary to the nucleic acid sequence of an apelin or an isoform thereof. The nucleic acid sequence of the apelin or an isoform thereof or the probe can be a ribonucleic acid (RNA, for example, an mRNA) sequence.

In yet another aspect of a method according to the invention, the detecting relying on the measurement of nucleotide expression is focused on the variation in apelin genomic nucleotide sequence (genotyping). The resulting genotype is, in one aspect, predictive of subsequent levels of apelin (and, thus, a disorder associated with a cardiac arrhythmia, such as atrial fibrillation, or a predisposition therefor).

In one embodiment of the invention, the disorder is atrial fibrillation. The atrial fibrillation can be, but is not limited to, lone atrial fibrillation.

In another embodiment of the invention, the apelin is apelin-12 (Tatemoto, K., et al., (1998) Biochem. Biophys. Res. Commun. 251 (2), 471-476). Other known isoforms of apelin contemplated for the methods described herein include, without limitation, apelin-36, apelin-31, apelin-28, apelin-17, and apelin-13 (D K Lee et al. J. Neurochem 2000; 73:34-41). In yet another aspect, the invention provides a method of identifying a subject having or at risk of having atrial fibrillation comprising measuring the amount of an apelin polypeptide, or a fragment thereof, in the plasma or a preparation thereof of the subject, and determining that the amount is reduced in comparison to that of a control subject, thereby identifying the subject having or at risk of having atrial fibrillation. By “at risk of having atrial fibrillation”, it is meant that the subject may not yet have had an episode of arrhythmia but, rather, is predisposed to arrhythmia. The measuring can comprise contacting the plasma or a preparation thereof with an immunological agent that binds with specificity to the apelin polypeptide or fragment thereof. The measuring can additionally comprise determining the amount of expression of the apelin polypeptide or a fragment thereof, for example, by contacting the plasma or a preparation thereof with at least one nucleic acid probe having a nucleic acid sequence complementary to apelin or an isoform thereof.

In yet another aspect, the invention provides kits for detecting a disorder associated with cardiac arrhythmia.

In one embodiment, the invention provides a kit for detecting a disorder associated with cardiac arrhythmia comprising an immunological agent that binds with specificity to an apelin polypeptide or a fragment thereof and instructions for use in accordance with the methods of the invention. The kit can further comprise a means for quantitating the amount of the apelin polypeptide, or fragment thereof, in a plasma sample or a preparation thereof obtained from a subject having a disorder associated with cardiac arrhythmia and for determining that the amount is reduced in comparison to a control sample.

In another embodiment, the invention provides a kit for detecting a disorder associated with cardiac arrhythmia comprising a means for determining the amount of expression of the apelin polypeptide or a fragment thereof and instructions for use in accordance with the methods of the invention. The means can comprise at least one nucleic acid probe having a nucleic acid sequence complementary to the nucleic acid sequence of an apelin or an isoform thereof and a means for determining that the amount of the nucleic acid sequence is reduced in comparison to a control sample.

In yet another aspect, the invention provides an article of manufacture for diagnosing a predisposition for atrial fibrillation in a subject comprising packaging material, a diagnostic kit, and instructions with the packaging material, wherein the diagnostic kit comprises an immunological agent that binds with specificity to the apelin polypeptide, or a fragment thereof, to form a bound complex and a means for measuring the quantity of bound complexes in a biological sample or a preparation thereof of a subject, wherein the quantity of bound complex as compared to a normal control is indicative for a predisposition for atrial fibrillation, and wherein the instructions indicate that the diagnostic kit can be used to diagnose a predisposition for atrial fibrillation in the subject.

These and other aspects of the invention, as well as various advantages and utilities, will be more apparent with reference to the detailed description of the preferred embodiments.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 depicts mean apelin-12 levels in subjects with lone atrial fibrillation as compared to matched controls.

FIG. 2 depicts a plot of the receiver operating characteristic (ROC) curve for the reciprocal of apelin-12.

FIG. 3 depicts the amino acid sequence for full-length human apelin as described by Tatemoto, K., et al., (1998) Biochem. Biophys. Res. Commun. 251 (2), 471-476 and as entered in the National Center for Biotechnology protein database under accession no. Q9ULZ1.

FIG. 4 depicts the nucleotide sequence for full-length human apelin as described by Tatemoto, K., et al., (1998) Biochem. Biophys. Res. Commun. 251 (2), 471-476.

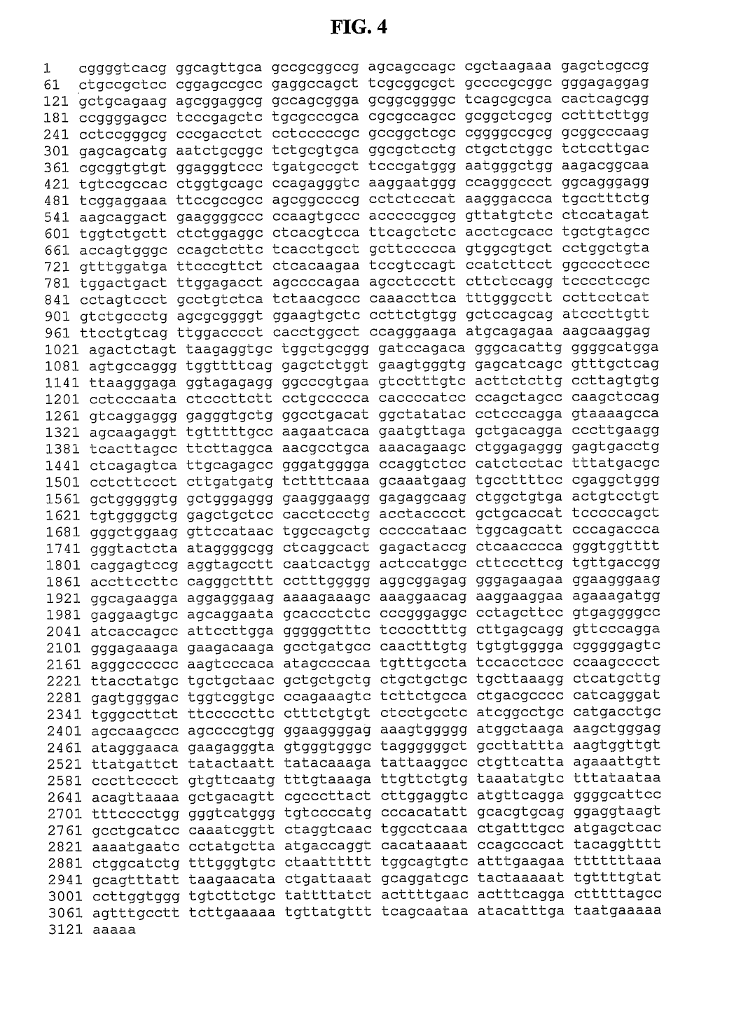

### DETAILED DESCRIPTION OF THE INVENTION

### I. Definitions

Unless defined otherwise, all technical and scientific terms used herein have the meaning commonly understood by a person skilled in the art to which this invention belongs. The following references provide one of skill with a general definition of many of the terms used in this invention: Lackie and Dow, The Dictionary of Cell & Molecular Biology (3^{rd} ed. 1999); Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “cardiac arrhythmia” is a rhythmic disturbance of the heartbeat, and can include such arrhythmias as those which cause hemodynamic upset (e.g., bradycardias and tachycardias).

“Atrial fibrillation” is a disorder in which the two atrial chambers of the heart fail to beat effectively. Consequently, blood is not pumped completely out of the chambers, so it may pool and clot.

“Stroke” is an infarction of brain tissue manifested by neurologic effects of varying severity.

“Congestive heart failure” is a disorder characterized by ineffective mechanical performance of the heart resulting in cardiac output inadequate to meet the body's needs.

As used herein, “plasma” refers to the fluid, noncellular portion of the blood, distinguished from the serum obtained after coagulation.

As used herein, “subject” refers to any warm-blooded animal, particularly including a member of the class Mammalia such as, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female.

As used herein, a “control subject” is a subject recruited from a healthy outpatient population who has been matched on the basis of age, sex, race, and ethnicity.

As used herein, “treatment” refers to ameliorating an adverse cardiac condition such as atrial fibrillation or congestive heart failure.

As used herein, “detecting” refers to methods which include identifying the presence or absence of substance(s) in the sample, quantifying the amount of substance(s) in the sample, and/or qualifying the type of substance. “Detecting” likewise refers to methods which include identifying the presence or absence of atrial fibrillation or congestive heart failure in a subject.

As to be used interchangeably herein, the terms “polynucleotide”, “nucleotides” and “oligonucleotides” refer to a polymeric form of nucleotides of any length, either deoxyribonucleotides or ribonucleotides, or analogs thereof. Non-limiting examples of polynucleotides include coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, a “polypeptide” refers to a chain of amino acids joined by peptide bonds.

As used herein, a “gene” refers to a polynucleotide containing at least one open reading frame that is capable of encoding a particular protein after being transcribed and translated.

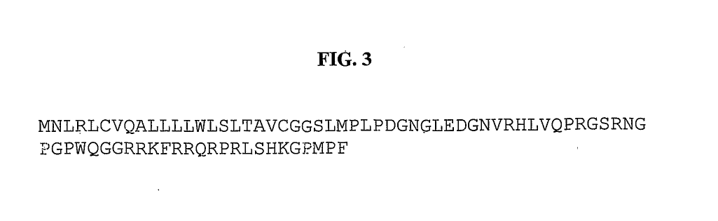

As used herein, an “isoform” refers to a peptide that is the product of the same gene. It can be a splice variant of the apelin gene or a post-translational processing variant, e.g., cleavage fragment, of the full-length apelin amino acid sequence. Isoforms of apelin described herein include, without limitation, apelin-36, apelin-31, apelin-28, apelin-17, and apelin-13 (D K Lee et al. J. Neurochem 2000; 73:34-41) and apelin-12 (Tatemoto, K., et al., (1998) Biochem. Biophys. Res. Commun. 251 (2), 471-476). These apelin isoforms are the products of processing to a specific number of carboxy-terminal (C-terminal) amino acids. For example, apelin-12 corresponds to the 12 C-terminal amino acids of the full-length apelin amino acid sequence depicted in FIG. 3. Apelin-13 corresponds to the 13 C-terminal amino acids of the full-length apelin amino acid sequence depicted in FIG. 3. Apelin-17 corresponds to the 17 C-terminal amino acids of the full-length apelin amino acid sequence depicted in FIG. 3. Apelin-28 corresponds to the 28 C-terminal amino acids of the full-length apelin amino acid sequence depicted in FIG. 3. Apelin-31 corresponds to the 31 C-terminal amino acids of the full-length apelin amino acid sequence depicted in FIG. 3. Apelin-36 corresponds to the 36 C-terminal amino acids of the full-length apelin amino acid sequence depicted in FIG. 3. Other as yet unidentified C-terminal (or other) processing products of the full-length apelin amino acid sequence may be relevant to the methods described herein.

As used herein, “differentially expressed”, as applied to nucleotide sequence or polypeptide sequence in a subject, refers to over-expression or under-expression of that sequence when compared to that detected in a control. Underexpression also encompass absence of expression of a particular sequence as evidenced by the absence of detectable expression of in a test subject when compared to a control.

The terms “comprises”, “comprising”, and the like are intended to have the broad meaning ascribed to them in U.S. Patent Law and can mean “includes”, “including” and the like.

It is to be understood that this invention is not limited to the particular component parts of a device described or process steps of the methods described, as such devices and methods may vary. It is also to be understood that the terminology used herein is for purposes of describing particular embodiments only, and is not intended to be limiting. As used in the specification and the appended claims, the singular forms “a”, “an”, and “the” include plural referents unless the context clearly indicates otherwise.

Other definitions appear in context throughout this disclosure.

### II. Methods of Apelin Detection

Diagnostic methods of the invention are based on detecting and/or monitoring reduced plasma levels of apelin. Preferably, the apelin is apelin-12.

A. Protein Expression

Expression of an apelin polypeptide or fragment thereof in a subject can be assessed immunologically, for example by Western blots, immunoassays such as radioimmunoprecipitation, enzyme-linked immunoassays, competitive immunoassays, bead agglomeration assays, sandwich-type immunoassays, such as ELISA, and the like.

Immunological agents can be employed in such assays. Immunological agents are typically antibodies or antigenic fragments. Antibodies of the invention comprise whole native antibodies, bispecific antibodies; chimeric antibodies; Fab, Fab′, single chain V region fragments (scFv) and fusion polypeptides. Fab fragments retain an entire light chain, as well as one-half of a heavy chain, with both chains covalently linked by the carboxy terminal disulfide bond. Fab fragments are monovalent with respect to the antigen-binding site. Preferably, the antibodies of the invention are monoclonal. The term “immunological agent” as used herein therefore includes intact immunoglobulin molecules as well as fragments thereof, such as Fab and Fab′, which are capable of binding to an apelin epitopic determinant.

The immunological agent should bind with specificity to the apelin epitopic determinant. “Binding with specificity” means that non-apelin polypeptides are either not specifically bound by the immunological agent or are only poorly recognized by the immunological agent.

Fragments and derivatives of apelin polypeptide sequences which would be expected to retain an epitopic determinant in whole or in part and are useful for immunological methodologies can be easily made by those skilled in the art given. An apelin polypeptide or fragment thereof should be immunogenic (e.g., containing an epitopic determinant) whether it results from the expression of the entire gene sequence, a portion of the gene sequence, or from two or more gene sequences which are ligated to direct the production of chimeric proteins. This reactivity can be demonstrated by standard immunological techniques, such as radioimmunoprecipitation, radioimmune competition, or immunoblots.

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology”, “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Antibodies are most conveniently obtained from hybridoma cells engineered to express an antibody. Methods of making hybridomas are well known in the art. The hybridoma cells can be cultured in a suitable medium, and spent medium can be used as an antibody source. Polynucleotides encoding the antibody can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Another method of obtaining antibodies is to immunize suitable host animals with an antigen and to follow standard procedures for polyclonal or monoclonal production. Monoclonal antibodies (Mabs) thus produced can be “humanized” by methods known in the art. Examples of humanized antibodies are provided, for instance, in U.S. Pat. Nos. 5,530,101 and 5,585,089.

“Humanized” antibodies are antibodies in which at least part of the sequence has been altered from its initial form to render it more like human immunoglobulins. In one another version, the heavy chain and light chain C regions are replaced with human sequence. In another version, the CDR regions comprise amino acid sequences for recognition of antigen of interest, while the variable framework regions have also been converted to human sequences. See, for example, EP 0329400. In a third version, variable regions are humanized by designing consensus sequences of human and mouse variable regions, and converting residues outside the CDRs that are different between the consensus sequences. The invention encompasses humanized Mabs. The invention also encompasses hybrid antibodies, in which one pair of heavy and light chains is obtained from a first antibody, while the other pair of heavy and light chains is obtained from a different second antibody. Such hybrids may also be formed using humanized heavy and light chains.

Construction of phage display libraries for expression of antibodies, particularly the Fab or scFv portion of antibodies, is well known in the art (Heitner, 2001). The phage display antibody libraries that express antibodies can be prepared according to the methods described in U.S. Pat. No. 5,223,409 incorporated herein by reference. Procedures of the general methodology can be adapted using the present disclosure to produce antibodies of the present invention. The method for producing a human monoclonal antibody generally involves (1) preparing separate heavy and light chain-encoding gene libraries in cloning vectors using human immunoglobulin genes as a source for the libraries, (2) combining the heavy and light chain encoding gene libraries into a single dicistronic expression vector capable of expressing and assembling a heterodimeric antibody molecule, (3) expressing the assembled heterodimeric antibody molecule on the surface of a filamentous phage particle, (4) isolating the surface-expressed phage particle using immunoaffinity techniques such as panning of phage particles against a preselected antigen, thereby isolating one or more species of phagemid containing particular heavy and light chain-encoding genes and antibody molecules that immunoreact with the preselected antigen.

Single chain variable region fragments are made by linking light and heavy chain variable regions by using a short linking peptide. Any peptide having sufficient flexibility and length can be used as a linker in a scFv. Usually the linker is selected to have little to no immunogenicity. An example of a linking peptide is (GGGGS)₃, which bridges approximately 3.5 nm between the carboxy terminus of one variable region and the amino terminus of another variable region. Other linker sequences can also be used. All or any portion of the heavy or light chain can be used in any combination. Typically, the entire variable regions are included in the scFv. For instance, the light chain variable region can be linked to the heavy chain variable region. Alternatively, a portion of the light chain variable region can be linked to the heavy chain variable region, or a portion thereof. Compositions comprising a biphasic scFv could be constructed in which one component is a polypeptide that recognizes an antigen and another component is a different polypeptide that recognizes a different antigen, such as a T cell epitope.

ScFvs can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect or mammalian cells, or prokaryotic, such as Escherichia coli, and the protein expressed by the polynucleotide can be isolated using standard protein purification techniques.

A particularly useful system for the production of scFvs is plasmid pET-22b(+) (Novagen, Madison, Wis.) in E. coli. pET-22b(+) contains a nickel ion binding domain consisting of 6 sequential histidine residues, which allows the expressed protein to be purified on a suitable affinity resin. Another example of a suitable vector for the production of scFvs is pcDNA3 (Invitrogen, San Diego, Calif.) in mammalian cells, described above.

Expression conditions should ensure that the scFv assumes functional and, preferably, optimal tertiary structure. Depending on the plasmid used (especially the activity of the promoter) and the host cell, it may be necessary or useful to modulate the rate of production. For instance, use of a weaker promoter, or expression at lower temperatures, may be necessary or useful to optimize production of properly folded scFv in prokaryotic systems; or, it may be preferable to express scFv in eukaryotic cells.

Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

The immunological agent can be immobilized on a solid surface. The immunological agent can be bound to a detectable label. The detectable label can be an enzyme label, or a fluorogenic compound. The binding site for the detectable label can be biotin, avidin or streptavidin. The immunological agent can be labeled radioisotopically, e.g., by ¹²⁵I, or conjugated directly to a detector enzyme, e.g., alkaline phosphatase or horse radish peroxidase, or can be labeled indirectly with a binding site for a detectable label, e.g., via biotinylation. The biotinylated immunological agent can then be detected by its ability to bind to a an avidin-linked enzyme. If the second immunological agent is biotinylated, a detector enzyme conjugated to avidin will be subsequently added.

The labels used in the assays of an embodiment of the invention can be primary labels (where the label comprises an element which is detected directly) or secondary labels (where the detected label binds to a primary label, e.g., as is common in immunological labeling). An introduction to labels, labeling procedures and detection of labels is found in Polak and Van Noorden (1997) Introduction to Immunocytochemistry, second edition, Springer Verlag, N.Y. and in Haugland (1996) Handbook of Fluorescent Probes and Research Chemicals, a combined handbook and catalogue Published by Molecular Probes, Inc., Eugene, Oreg. Primary and secondary labels can include undetected elements as well as detected elements.

Useful primary and secondary labels in one embodiment of the present invention can include spectral labels such as fluorescent dyes (e.g., fluorescein and derivatives such as fluorescein isothiocyanate (FITC) and Oregon Green™, rhodamine and derivatives (e.g., Texas red, tetramethylrhodamine isothiocyanate (TRITC), etc.), digoxigenin, biotin, phycoerythrin, AMCA, CyDyes™, and the like), radiolabels (e.g., ³H, ¹²⁵I, 35S, ¹⁴C, ³²P, ³³P), enzymes (e.g., horse-radish peroxidase, alkaline phosphatase) spectral colorimetric labels such as colloidal gold or colored glass or plastic (e.g. polystyrene, polypropylene, latex) beads. The label can be coupled directly or indirectly to a component of the detection assay (e.g., the labeling nucleic acid) according to methods well known in the art. As indicated above, a wide variety of labels can be used, with the choice of label depending on sensitivity required, ease of conjugation with the compound, stability requirements, available instrumentation, and disposal provisions. In general, a detector which monitors an analyte-receptor complex is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound analyte is digitized for subsequent computer analysis.

In specific embodiments of the invention, preferred labels include those which utilize 1) chemiluminescence (using Horseradish Peroxidase and/or Alkaline Phosphatase with substrates that produce photons as breakdown products) with kits being available, e.g., from Molecular Probes, Amersham, Boehringer-Mannheim, and Life Technologies/Gibco BRL; 2) color production (using both Horseradish Peroxidase and/or Alkaline Phosphatase with substrates that produce a colored precipitate) (kits available from Life Technologies/Gibco BRL, and Boehringer-Mannheim); 3) hemifluorescence using, e.g., Alkaline Phosphatase and the substrate AttoPhos (Amersham) or other substrates that produce fluorescent products, 4) Fluorescence (e.g., using Cy-5 (Amersham), fluorescein, and other fluorescent tags); 5) radioactivity using kinase enzymes or other approaches. Other methods for labeling and detection will be readily apparent to one skilled in the art.

B. Nucleotide Expression

The expression profile of apelin can be a protein profile or it can be an RNA (e.g., mRNA) profile. Thus, expression of an apelin polypeptide or fragment thereof in a subject can be assessed, for example by measuring the amount of an apelin nucleic acid sequence, such as a ribonucleic acid (RNA) sequence (e.g., mRNA) using a complementary nucleic acid sequence as a probe.

The complementary nucleic acid sequence may, but need not necessarily be, 100% complementary to the nucleic acid sequence of an apelin or an isoform thereof, but, rather, must be substantially similar to the nucleic acid sequence of an apelin or an isoform thereof to allow detection of the desired apelin nucleic acid of interest. The nucleic acid sequence may be 60-100% complementary to the nucleic acid sequence of an apelin or an isoform thereof. In a further embodiment, the sequence is 85-100% complementary to the nucleic acid sequence of an apelin or an isoform thereof. In still a further embodiment, the sequence is 95-100% complementary to the nucleic acid sequence of an apelin or an isoform thereof. The difference in percentage lies in the number of nucleic acid residues that are complementary base-pair matches (e.g., A-T, G-C).

Accordingly, an adequately complementary nucleic acid sequence or probe of a method of the invention hybridizes under stringent conditions to a nucleic acid sequence encoding the amino acid sequence of apelin or an isoform thereof. Preferably, the sequence hybridizes under highly stringent conditions. As used herein, the term “hybridizes under stringent conditions” is intended to describe conditions for hybridization and washing under which nucleotide sequences at least 60%, 85%, or 95% homologous to each other typically remain hybridized to each other. Hybridization conditions are known to those skilled in the art and can be found in Current Protocols in Molecular Biology, John Wiley & Sons, N.Y., 6.3 α subunit.1-6.3 α subunit.6, 1991.

Depending upon what type of expression product is being analyzed, the method of analysis and quantitation of the expression product will differ. If the nucleic acid expression product is itself a nucleic acid, such as an RNA (e.g., mRNA), then it can be quantitated using a number of methods including but not limited to Northern analysis, reverse-transcriptase polymerase chain reaction (RT-PCR), and gene expression/cDNA microarray analysis. These techniques can rapidly identify genes that are up- or down-regulated in different samples or in response to specific stimuli and have been reported in the literature, and, thus, one of ordinary skill will be familiar with these. (See, for example, Methods Enzymol 303:349-380, 1999; Ying and Lin in Biotechniques 26:966-8, 1999; Zhao et al., J Biotechnol 73:35-41, 1999; and Blumberg and Belmonte in Methods Mol Biol 97:555-574, 1999.) In some embodiments, the nucleic acids are harvested from the circulating cells or plasma and analyzed without the need for in vitro amplification.

DNA microarrays can measure expression by using templates containing apelin probes that are exposed simultaneously to a target sample, allowing a systematic survey of DNA and RNA variation. Quantitative monitoring of gene expression patterns with a complementary DNA microarray is described in Schena et al. (1995) Science 270:467. Expression analysis using nucleic acid arrays is reviewed by Ramsay (1998) Nat. Biotech. 16:40-44. Methods for creating microarrays of biological samples are described in U.S. Pat. Nos. 5,807,522 and 5,445,934.

Array-based technology involves hybridization of a pool of target polynucleotides corresponding to gene transcripts of a test subject to an array of apelin probe sequences immobilized on the array substrate. The technique allows simultaneous detection of multiple gene transcripts and yields quantitative information on the relative abundance of each gene transcript expressed in a test subject. By comparing the hybridization patterns generated by hybridizing different pools of target polynucleotides to the arrays, one can readily obtain the relative transcript abundance in two pools of target samples. The analysis can be extended to detecting differential expression of apelin between diseased and normal tissues, among different types of tissues and cells, amongst cells at different cell-cycle points, or at different developmental stages, and amongst cells that are subjected to various environmental stimuli or lead drugs.

A particularly important application of the microarray method allows for the assessment of differential gene expression in pairs of different mRNA samples (from different subjects), or in the same subject comparing normal versus disease states or time progression of the disease. Microarray analysis allows one to analyze the expression of apelin.

Identification of the differentially expressed apelin as in the present invention can, for example, be performed by: constructing normalized and subtracted cDNA libraries from mRNA extracted from circulating cell or plasma samples of healthy subjects and diseased subjects; purifying the DNA of clones from cDNA libraries representing healthy and diseased circulating cell or plasma samples, microarraying the purified DNA for expression analysis; and probing microarrays to identify the genes from the clones that are differentially expressed using labeled cDNA from healthy and diseased circulating cell or plasma samples.

In a specific embodiment of the microarray technique, PCR-amplified inserts of cDNA clones are applied to a substrate in a dense array. The microarrayed genes, immobilized on the microchip, are suitable for hybridization under stringent conditions. Hybridization can be performed under conditions of different “stringency”. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form.

Fluorescently labeled cDNA probes may be generated through incorporation of fluorescent nucleotides by reverse transcription of RNA extracted from samples of interest. Labeled cDNA probes applied to the chip hybridize with specificity to each spot of DNA on the array. After stringent washing to remove non-specifically bound probes, the chip is scanned by confocal laser microscopy. Quantitation of hybridization of arrayed apelin allows for assessment of corresponding mRNA abundance. With dual color fluorescence, separately labeled cDNA probes generated from two sources of RNA are hybridized pairwise to the array. The relative abundance of the transcripts from the two sources corresponding to apelin is, thus, determined simultaneously. The technique has been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(20):106-49 (1996)). As a result, genes which are differentially expressed in normal and diseased sample, are revealed and can be further identified by DNA sequencing.

Once potentially differentially expressed apelin sequences have been identified using techniques such as those described above, the differential expression of such putatively, differentially expressed genes may be corroborated. Corroboration can be accomplished via, for example, such well-known techniques as Northern analysis, quantitative RT-coupled PCR, microarrays, or RNase protection.

Other detection/quantitation methods based on nucleotide expression that are contemplated herein include, without limitation, genotyping (e.g., to determine genetic variation at the apelin locus) and real-time polymerase chain reaction.

In addition to, or in conjunction with the correlation of expression profiles and clinical data, it is often desirable to correlate expression patterns with the subject's genotype at least one genetic locus or to correlate both expression profiles and genetic loci data with clinical data.

Numerous well known methods exist for evaluating the genotype of an individual, including Southern analysis, restriction fragment length polymorphism (RFLP) analysis, polymerase chain reaction (PCR), amplification length polymorphism (AFLP) analysis, single stranded conformation polymorphism (SSCP) analysis, single nucleotide polymorphism (SNP) analysis (e.g., via PCR, Taqman or molecular beacons), among many other useful methods. Many such procedures are readily adaptable to high throughput and/or automated (or semi-automated) sample preparation and analysis methods. Most, can be performed on nucleic acid samples recovered via simple procedures from the same sample of leukocytes as yielded the material for expression profiling. Exemplary techniques are described in, e.g., in Ausubel, et al. (supplemented-through 2000) Current Protocols in Molecular Biology John Wiley & Sons, New York (“Ausubel”); Sambrook, et al. 1989 Molecular Cloning-A Laboratory Manual (2nd Ed.), Vol. 1-3, Cold Spring Harbor Laboratory.

Real-time reverse-transcriptase (RT) polymerase chain reaction (PCR) quantitates the initial amount of the template most specifically, sensitively and reproducibly and provides an alternative to other forms of quantitative RT-PCR that detect the amount of final amplified product at the end-point (Freeman, W. M., et al. 1999 Biotechniques 26(1):112-22, 124-5; Raeymaekers, L. 2000 Mol Biotechnol 15(2):115-22). Real-time PCR monitors the fluorescence emitted during the reaction as an indicator of amplicon production during each PCR cycle (i.e., in real time) as opposed to the endpoint detection (Higuchi, R., et al. 1992 Biotechnology (NY) 10(4):413-7; Higuchi, R., et al. 1993 Biotechnology (NY) 11(9):1026-30).

The real-time PCR system is based on the detection and quantitation of a fluorescent reporter (Lee, L. G., et al. 1993 Nucleic Acids Res 21(16):3761-6; Livak, K. J., et al. 1995 PCR Methods Appl 4(6):357-62). This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. A significant increase in fluorescence above the baseline value measured during the 3-15 cycles indicates the detection of accumulated PCR product.

### III. Kits

An additional embodiment of the invention provides a kit for use in the diagnosis of disorders associated with cardiac arrhythmia. Such kits can comprise an immunological agent that binds with specificity to a polypeptide encoding apelin or a fragment thereof and instructions for its use. Kits of the invention can further comprise reagents necessary for visualization or quantification. These reagents can include specific antibodies capable of identifying apelin or its gene products, other antibodies, markers and standards that are needed for visualization or quantification, as well as buffers, diluents, washing solutions and the like, commonly contained in a commercial reagent kit.

Kits containing the polynucleotide probe arrays described herein are likewise contemplated. Such kits allow simultaneous detection of the expression and/or quantification of the level of expression of gene transcripts of a subject. Further embodied by the invention are kits useful for detecting differential expression of gene transcripts of a test subject in comparison to a control subject.

Such kits may comprise the reagents which render the hybridization procedure possible: an array of polynucleotide probes of the invention used for detecting target polynucleotides; hybridization reagents that allow formation of stable target-probe complexes during a hybridization reaction. The kits may also contain reagents useful for generating labeled target polynucleotides corresponding to gene transcripts of a test subject. Optionally, the arrays contained in the kits may be pre-hybridized with polynucleotides corresponding to gene transcripts of the control to which the test subject is compared.

Each reagent can be supplied in a solid form or dissolved/suspended in a liquid buffer suitable for inventory storage, and later for exchange or addition into the reaction medium when the test is performed. Suitable packaging is provided. The kit can optionally provide additional components that are useful in the procedure. These optional components include, but are not limited to, buffers, capture reagents, developing reagents, labels, reacting surfaces, means for detection, control samples, instructions, and interpretive information. The kits can be employed to test a variety of biological samples, including body fluid, solid tissue samples, circulating cells, tissue cultures or cells derived therefrom and the progeny thereof, and sections or smears prepared from any of these sources. Diagnostic or prognostic procedures using the kits of this invention can be performed by clinical laboratories, experimental laboratories, practitioners, or private individuals.

Thus, a kit can be contemplated for detecting a disorder associated with a cardiac arrhythmia comprising an immunological agent that binds with specificity to an apelin polypeptide or a fragment thereof and instructions for use in accordance with the methods of the invention. The kit can further comprise a means for quantitating the amount of the apelin polypeptide, or fragment thereof, in a plasma sample obtained from a subject having a cardiac arrhythmia and determining that the amount is reduced in comparison to a control sample.

Additionally, a kit can be contemplated for detecting a disorder associated with a cardiac arrhythmia comprising a means for determining the amount of expression of the apelin polypeptide or a fragment thereof and instructions for use in accordance with the methods of the invention. The means can comprise at least one nucleic acid probe having a nucleic acid sequence complementary to the nucleic acid sequence of an apelin or an isoform thereof and a means for determining that the amount of the nucleic acid sequence is reduced in comparison to a control sample.

Additionally, an article of manufacture may be contemplated for diagnosing a predisposition for atrial fibrillation, stroke, or congestive heart failure in a subject comprising packaging material, a diagnostic kit, and instructions within the packaging material, wherein the diagnostic kit comprises an immunological agent the binds with specificity to the apelin polypeptide, or a fragment thereof, to form a bound complex and a means for measuring the quantity of bound complexes in a biological sample from a subject, wherein the quantity of bound complex as compared to a normal control is indicative for a predisposition for atrial fibrillation, stroke, or congestive heart failure, and wherein the instructions indicate that the diagnostic kit can be used to diagnose a predisposition for atrial fibrillation, stroke, or congestive heart failure in the subject.

Those skilled in the art will recognize, or be able to ascertain using no more than routine experimentation, many equivalents to the specific embodiments of the invention described herein. Such equivalents are intended to be encompassed by the following claims. All references disclosed herein are incorporated by reference in their entirety.

The invention will be more fully understood by reference to the following examples. These examples, however, are merely intended to illustrate the embodiments of the invention and are not to be construed to limit the scope of the invention.

### EXAMPLES

### Example 1

### Reduction of Plasma Apelin Levels in Subjects with Atrial Fibrillation

There is some evidence of a subtle underlying atrial or ventricular myopathy even in lone AF (A. Frustaci et al. Cardiologia 1997; 96:1180-4 and A. Fruustaci et al. Circulation 1997; 96:3157-63). However, the relationship between AF and myocardial abnormalities has been obscured to some extent by the rapid cellular remodeling seen with the arrhythmia. (J. Ausma et al. Circulation. 1997; 96:3157-63; B J. Brundel et al. Cardiovasc. Res. 2002; 54:315-24; S, Nattel et al. Nature 2002; 415:219-26) It was hypothesized that there might be abnormalities in atrial endocrine function early in the course of primary forms of AF and therefore, the humoral axes involved in signaling to and from atrial cells was explored. One such endocrine axis is the recently described apelin-angiotensin receptor-like 1(APJ) pathway (K. Tatemoto et al. Biochem Biophys Res Commun 1998; 251:471-6) Apelin levels were studied in 73 subjects with lone Atrial fibrillation.

In a homogeneous group of patients with lone AF, a highly significant reduction of plasma apelin levels was observed when compared to a matched control population. Apelin-12 levels also were inversely correlated with levels of nt-proBNP. These findings confirm a subtle but definite perturbation of the cardiac humoral axis in individuals with a history of AF but without overt structural heart disease.

Study subjects had electrocardiographic evidence of paroxysmal or chronic AF and a structurally normal heart on echocardiography. Controls were recruited from a healthy outpatient population. Plasma apelin levels were determined using a commercially available immunoassay.

Consent was obtained from each study subject. Individuals were considered eligible for enrollment if they had at least one documented EKG with AF and had a structurally normal heart on echocardiography. Subjects were excluded if they had a history of myocardial infarction, rheumatic heart disease, cardiomyopathy, significant valvular disease, hyperthyroidism or hypertension. Seventy-three subjects with lone AF were enrolled between Jul. 5, 2001 and Apr. 17, 2002. These subjects were matched on the basis of age, sex, race and ethnicity to 73 control subjects recruited from a healthy outpatient population (Genomics Collaborative Inc.).

Each subject underwent a physical examination and a structured interview to elicit details of symptoms, past medical history, medications, and possible triggers for AF. The medical history of all first-degree relatives was obtained using a standardized questionnaire. All subjects with lone AF underwent electrocardiography at enrollment, and a standardized echocardiogram was also obtained from each individual. This study included a comprehensive 2-D, M-mode and Doppler evaluation. Ejection fraction was estimated using the modified Quinones method (A E. Weyman Principles and practice of echocardiography 1993). Blood samples for genetic and serologic analyses were drawn at enrollment from each subject in the sitting position.

Blood samples were obtained in EDTA containing tubes and centrifuged. Plasma was extracted, aliquoted, and stored at −80 degrees Celsius until analysis. Plasma apelin-12 levels were determined using a commercially available enzyme immunoassay without extraction (Phoenix Pharmaceuticals, Belmont, Calif.) according to the manufacturer's instructions. This assay employs an immunoaffinity purified rabbit antibody specific for apelin 1-12. The antibody has 100% cross reactivity to apelin 1-12, 1-13 and 1-36; there is no cross reactivity to ADM-52, BNP-32, CNP-22, ANP (25-56), ghrelin, ET-1, or bradykinin. Plasma proANP and nt-proBNP levels were determined using commercially available enzyme immunoassays without extraction (distributed in the United States by ALPCO diagnostics, Windham, N.H., and manufactured by Biomedica Gruppe, Germany) according to the manufacturer's instructions. All assays were performed in duplicate with intra-experimental standards using a Victor 3 plate reader (Perkin-Elmer, Wellesley, Mass.). Values were normalized to a standard curve. The intra-assay and inter-assay variances for apelin-12 were 24 and 18%, respectively.

Plasma apelin-12 values were highly skewed and therefore, were log transformed prior to analysis. Normally distributed values are displayed as means with 95% confidence intervals. For comparisons between lone AF and control populations, cases were paired with healthy controls matched for age, gender, race and ethnicity. The means of normally distributed continuous variables were compared using paired t-tests. Differences between groups for categorical variables were compared using a Chi-squared or Fisher's exact test.

In subjects with lone AF, a multivariate analysis was performed to determine the correlates of apelin-12 levels by regressing log-transformed apelin-12 values on clinical variables with a p value of <0.1 on univariate analysis. Data were compiled and analyzed in MS Excel 2000, MS Access 2000 (Microsoft Office 2000, Redmond, Wash., USA), and Intercooled Stata 8.0 (Stata Corp, College Station, Tex., USA).

During the study period a total of seventy-three subjects with lone AF were enrolled. Seventy-three healthy control subjects were matched to subjects with lone AF based on age, gender, race and ethnicity. Body mass index, systolic and diastolic blood pressures were similar between subjects and controls (Table 1).

TABLE 1
Baseline characteristics of subjects with lone atrial fibrillation and controls.

| Baseline Characteristics | Lone AF | | Controls | |
|---|---|---|---|---|
| Number | 73 | | 73 | |
| Male | 79.5% | (58) | 79.5% | (58) |
| Age at enrollment | 54.2 | (CI 51.9-56.5) | 54.3 | (CI 51.8-56.7) |
| BMI | 26.5 | (CI 25.5-27.4) | 26.9 | (CI 25.9-28.0) |
| Systolic Blood Pressure, mmHg | 121.5 | (CI 118.4-124.6) | 126.6 | (123.1-130.0) |
| Diastolic Blood Pressure, mmHg | 75.0 | (CI 73.2-76.7) | 77.7 | (74.8-80.6) |
| Race and Ethnicity | | | | |
| Caucasian | 97.3% | (71) | 97.3% | (71) |
| African-American | 1.4% | (1) | 1.4% | (1) |
| Asian | 1.4% | (1) | 1.4% | (1) |
| Medications | | | | |
| Beta-blocker | 53.4 | (39) | 0 | |
| Digoxin | 11.0 | (8) | 0 | |
| Calcium channel blocker | 19.2 | (14) | 0 | |
| Lipid lowering agent | 13.7 | (10) | 0 | |
| ACE inhibitor or ARB | 0 | | 0 | |
| Personal History of AF | | | | |

| | | | |
|---|---|---|---|
| Age at first diagnosis of AF | 47.1 | (CI 44.5-49.7) | — |
| Over 100 episodes AF | 53.1 | (34) | — |
| Paroxysmal AF at initial | 95.9 | (70) | — |
| presentation | | | |
| Paroxysmal AF at study enrollment | 89.0 | (65) | — |
| History of an electrical | 34.3 | (25) | — |
| cardioversion | | | |

The mean age at diagnosis with AF was 47.1±11.3 years, and mean age at enrollment was 54.2±10.1 years. As observed in other cohorts with lone AF, subjects were predominately male (79.5%). Ninety-six percent of subjects initially presented with paroxysmal AF, and 89% of subjects remained in paroxysmal AF at study enrollment (Table 2).

TABLE 2
Electrocardiographic and echocardiographic characteristics
of subjects with lone atrial fibrillation. Values are presented
as number (percentage) unless otherwise indicated.

| | |
|---|---|
| Electrocardiogram | |
| Normal sinus rhythm | 25 (34.3) |
| Sinus bradycardia | 33 (45.2) |
| Atrial fibrillation | 8 (11.0) |
| Atrial flutter | 3 (4.1) |
| Paced or other rhythm | 4 (5.5) |
| Mean ventricular rate | 62.6 (CI 58.8-66.4) bpm |
| P-R interval | 175 (CI 167-184) ms |
| QRS interval | 93.9 (CI 91.2-96.5) ms |
| QTc interval | 406 (CI 396-416) ms |
| Axis | 35.3 (CI 26-44.8) degrees |
| Left atrial enlargement | 16 (26.7) |
| Left ventricular hypertrophy | 2 (2.9) |
| Echocardiogram | |
| Ejection Fraction | 61.3 (CI 59.8-62.9) percent |
| Left atrial size | 39.4 (CI 37.8-41.1) mm |
| Left ventricular internal dimension | 49.9 (CI 48.7-51.2) mm |
| Aortic root | 33.1 (CI 31.4-34.8) mm |
| Posterior wall thickness | 9.8 (CI 9.4-10.0) mm |
| Interventricular septal wall thickness | 10.2 (CI 9.7-10.6) mm |

The majority of subjects with lone AF had frequent, paroxysmal arrhythmias with 53% reporting more than 100 lifetime episodes. While 27% of subjects in sinus rhythm had evidence of left atrial enlargement on EKG, there were no other electrocardiographic abnormalities. Echocardiography was notable for a mean left atrial diameter at the upper limits of normal. The mean values of all other echocardiographic parameters including chamber dimensions, wall thicknesses and functional indices were normal in the study cohort. Control subjects had no significant past medical history.

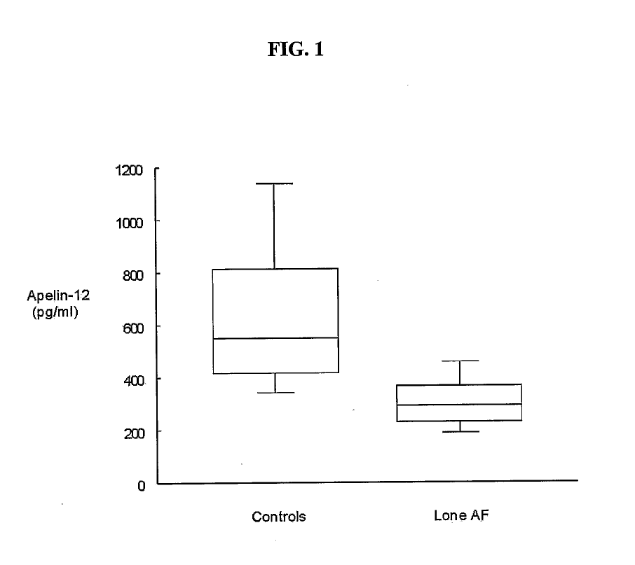

Mean apelin-12 levels were significantly decreased in subjects with lone AF when compared to matched controls (187 pg/ml versus 304 pg/ml, p<0.00005) (FIG. 1). As previously reported mean nt-proBNP levels were elevated [187 fmol/ml (CI 161-215) vs. 145 fmol/ml (CI 116-173), p=0.0016], but mean proANP levels were unchanged [2421 fmol/ml (CI 1421-3421) vs. 2166 fmol/ml (CI 1658-2673), p=0.52] in lone AF subjects when compared with controls. A strong negative correlation was observed between apelin-12 and nt-proBNP levels in subjects with lone AF (r=−0.32, p=0.005). A weak negative correlation was noted between systolic blood pressure and apelin-12 levels (r=−0.21, p=0.078).

The results of multivariable regression models using clinical variables are illustrated in Table 3.

TABLE 3
Predictors of log (apelin) from multivariable linear regression
among subjects with lone atrial fibrillation.

| | Clinical variable | | |
|---|---|---|---|
| | â | SE | P value |
| Systolic Blood Pressure | −0.002 | 0.001 | 0.107 |
| Log (nt-proBNP) | −0.184 | 0.072 | 0.012 |

Clinical variables with a p value of <0.1 on univariate analysis included log (nt-proBNP), and systolic blood pressure. A significant association was noted only between the log (apelin-12) and log (nt-proBNP) values (â=−0.184, SE=0.072, p=0.012). The proportion of variance in apelin-12 levels explained by the multivariable model was 0.36.

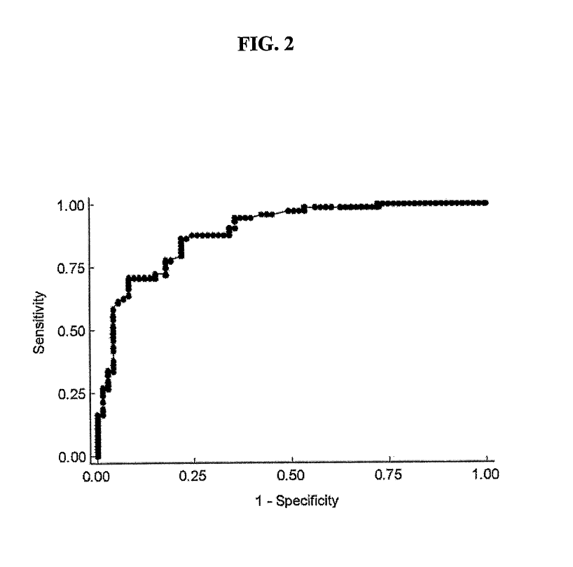

To assess the utility of apelin levels in discriminating between those with lone AF and normal controls, the receiver operating characteristic (ROC) curve for the reciprocal of apelin-12 in this context was plotted (FIG. 2). The area under the curve was 0.89 (CI 0.84-0.94).

Seventy-three subjects with lone AF and seventy-three healthy controls were enrolled and studied. Mean levels of apelin were significantly lower in subjects with LAF when compared to controls (307 pg/ml versus 648 pgl/ml, p<0.00005). Reduced apelin levels were observed in this homogenous population of lone AF subjects, representing an underlying diathesis predisposing to this common arrhythmia.

Apelin levels are significantly reduced in a cohort of well-characterized subjects with lone AF. These abnormalities of circulating apelin are present even when the study subjects are in sinus rhythm. In multivariable analysis only nt-proBNP was significantly correlated with apelin levels. These data extend previous observations that lone AF is associated with abnormal natriuretic peptide profiles. Atrial endocrine function may be disrupted in those who have had even a single episode of the arrhythmia (A. Rossi et al. J Am Coll Cardiol. 2000; 35:1256-62 and H. Silvet et al. Am J. Cardiol. 2003; 92:1124-7). These findings confirm a perturbation of the cardiac humoral axis in individuals with a history of AF but without overt structural heart disease.

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended numbered claims.

1. A method of detecting a disorder associated with a cardiac arrhythmia in a subject having said disorder comprising measuring the amount of an apelin polypeptide, or a fragment thereof, in the plasma of the subject, and determining that the amount is reduced in comparison to that of a control subject, thereby detecting said disorder.

2. The method of claim 1, wherein the disorder is atrial fibrillation.

3. The method of claim 2, wherein the atrial fibrillation is lone atrial fibrillation.

4. The method of claim 3, wherein the apelin polypeptide is apelin-12.

5. The method of claim 1, wherein the measuring comprises contacting the plasma or a preparation thereof with an immunological agent the binds with specificity to the apelin polypeptide or fragment thereof.

6. A method of detecting a disorder associated with a cardiac arrhythmia in a subject having said disorder comprising measuring the amount of an apelin nucleotide sequence in the circulating cells or plasma of the subject, and determining that the amount is reduced in comparison to that of a control subject, thereby detecting said disorder.

7. The method of claim 6, wherein the disorder is atrial fibrillation.

8. The method of claim 6, wherein the apelin polypeptide is apelin-12.

9. The method of claim 6, wherein the measuring comprises contacting the circulating cells or plasma or a preparation thereof with at least one nucleic acid probe having a nucleic acid sequence complementary to the nucleic acid sequence of apelin or an isoform thereof.

10. The method of claim 9, wherein the nucleic acid sequence of apelin or an isoform thereof is a ribonucleic acid (RNA) sequence.

11. A method of identifying a subject having or at risk of having atrial fibrillation comprising measuring the amount of an apelin polypeptide, or a fragment thereof, in the circulating cells or plasma or a preparation thereof of the subject, and determining that the amount is reduced in comparison to that of a control subject, thereby identifying the subject having or at risk of having atrial fibrillation.

12. The method of claim 11, wherein the atrial fibrillation is lone atrial fibrillation.

13. The method of claim 11, wherein the apelin polypeptide is apelin-12.

14. The method of claim 11, wherein the measuring comprises contacting the plasma or a preparation thereof with an immunological agent that binds with specificity to the apelin polypeptide or fragment thereof.

15. The method of claim 11, wherein the measuring comprises determining the amount of expression of the apelin polypeptide or a fragment thereof.

16. The method of claim 15, wherein determining the amount of expression comprises contacting the circulating cells or plasma or a preparation thereof with at least one nucleic acid probe having a nucleic acid sequence complementary to apelin or an isoform thereof.

17. A kit for detecting a disorder associated with cardiac arrhythmia comprising an immunological agent that binds with specificity to an apelin polypeptide or a fragment thereof and instructions for use according to claim 1.

18. The kit of claim 17, further comprising a means for quantitating the amount of the apelin polypeptide, or fragment thereof, in a plasma sample or preparation thereof obtained from a subject having a cardiac arrhythmia and for determining that the amount is reduced in comparison to a control sample.

19. A kit for detecting a disorder associated with cardiac arrhythmia comprising a means for determining the amount of expression of the apelin polypeptide or a fragment thereof and instructions for use according to claim 6.

20. The kit of claim 19, wherein the means comprises at least one nucleic acid probe having a nucleic acid sequence complementary to the nucleic acid sequence of an apelin or an isoform thereof.

21. The kit of claim 17 or 10, wherein the disorder is atrial fibrillation.

22. An article of manufacture for diagnosing a predisposition for atrial fibrillation in a subject comprising packaging material, a diagnostic kit, and instructions with the packaging material, wherein the diagnostic kit comprises an immunological agent that binds with specificity to the apelin polypeptide, or a fragment thereof, to form a bound complex and a means for measuring the quantity of bound complexes in a biological sample or a preparation thereof of a subject, wherein the quantity of bound complex as compared to a normal control is indicative for a predisposition for atrial fibrillation, and wherein the instructions indicate that the diagnostic kit can be used to diagnose a predisposition for atrial fibrillation in the subject.

23. The kit of claim 19, wherein the disorder is atrial fibrillation.


SEQUENCE LISTING (3 sequences)

SEQ ID NO: 1 - protein, 77 aa, Homo sapiens
MNLRLCVQALLLLWLSLTAVCGGSLMPLPDGNGLEDGNVRHLVQPRGSRNGPGPWQGGRRKFRRQRPRLSHKGPMPF

SEQ ID NO: 2 - DNA, 3125 bp, Homo sapiens
cggggtcacgggcagttgcagccgcggccgagcagccagccgctaagaaagagctcgccgctgccgctcccggagccgccgaggccagcttcgcggcgctgccccgcggcgggagaggaggctgcagaagagcggaggcggccagcgggagcggcggggctcagcgcgcacactcagcggccggggagcctcccgagctctgcgcccgcacgcgccagccgcggctcgcgcctttcttggcctccgggcgcccgacctctcctcccccgcgccggctcgccggggccgcggcggcccaaggagcagcatgaatctgcggctctgcgtgcaggcgctcctgctgctctggctctccttgaccgcggtgtgtggagggtccctgatgccgcttcccgatgggaatgggctggaagacggcaatgtccgccacctggtgcagcccagagggtcaaggaatgggccagggccctggcagggaggtcggaggaaattccgccgccagcggccccgcctctcccataagggacccatgcctttctgaagcaggactgaaggggcccccaagtgcccacccccggcggttatgtctcctccatagattggtctgcttctctggaggcctcacgtccattcagctctcacctcgcacctgctgtagccaccagtgggcccagctcttctcacctgcctgcttcccccagtggcgtgctcctggctgtagtttggatgattcccgttctctcacaagaatccgtccagtccatcttcctggcccctccctggactgactttggagacctagccccagaaagcctcccttcttctccaggtcccctccgccctagtccctgcctgtctcatctaacgccccaaaccttcatttgggccttccttcctcatgtctgccctgagcgcggggtggaagtgctcccttctgtgggctccagcagatcccttgttttcctgtcagttggacccctcacctggcctccagggaagaatgcagagaaaagcaaggagagactctagttaagaggtgctggctgcggggatccagacagggcacattgggggcatggaagtgccagggtggttttcaggagctctggtgaagtgggtggagcatcagcgtttgctcagttaagggagaggtagagaggggcccgtgaagtcctttgtcacttctcttgccttagtgtgcctcccaatactcccttcttcctgcccccacaccccatccccagctagcccaagctccaggtcaggaggggagggtgctgggcctgacatggctatataccctcccaggagtaaaagccaagcaagaggttgtttttgccaagaatcacagaatgttagagctgacaggacccttgaaggtcacttagccttcttaggcaaacgcctgcaaaacagaagcctggagaggggagtgacctgctcagagtcattgcagagccgggatggggaccaggtctcccatctcctactttatgacgccctcttccctcttgatgatgtcttttcaaagcaaatgaagtgccttttcccgaggctggggctgggggtggctgggaggggaagggaagggagaggcaagctggctgtgaactgtcctgttgtggggctggagctgctcccacctccctgacctacccctgctgcaccattcccccagctgggctggaaggttccataactggccagctgcccccataactggcagcattcccagacccagggtactctaataggggcggctcaggcactgagactaccgctcaaccccagggtggttttcaggagtccgaggtagccttcaatcactggactccatggccttcccttcgtgttgaccggaccttccttccagggcttttcctttgggggaggcggagaggggagaagaaggaagggaagggcagaaggaaggagggaagaaaagaaagcaaaggaacagaaggaaggaaagaaagatgggaggaagtgcagcaggaatagcaccctctccccgggaggccctagcttccgtgaggggccatcaccagccattccttggagggggctttctccccttttgcttgagcagggttcccaggagggagaaagagaagacaagagcctgatgcccaactttgtgtgtgtggggacgggggagtcagggccccccaagtcccacaatagccccaatgtttgcctatccacctcccccaagcccctttacctatgctgctgctaacgctgctgctgctgctgctgctgcttaaaggctcatgcttggagtggggactggtcggtgcccagaaagtctcttctgccactgacgcccccatcagggattgggccttctttcccccttcctttctgtgtctcctgcctcatcggcctgccatgacctgcagccaagcccagccccgtggggaaggggagaaagtgggggatggctaagaaagctgggagatagggaacagaagagggtagtgggtgggctaggggggctgccttatttaaagtggttgtttatgattcttatactaatttatacaaagatattaaggccctgttcattaagaaattgttcccttcccctgtgttcaatgtttgtaaagattgttctgtgtaaatatgtctttataataaacagttaaaagctgacagttcgcccttactcttggaggtcatgttcaggaggggcattcctttcccctgggggtcatgggtgtccccatgcccacatattgcacgtgcagggaggtaagtgcctgcatcccaaatcggttctaggtcaactggcctcaaactgatttgccatgagctcacaaaatgaatccctatgcttaatgaccaggtcacataaaatccagcccacttacaggttttctggcatctgtttgggtgtcctaatttttttggcagtgtcatttgaagaatttttttaaagcagtttatttaagaacatactgattaaatgcaggatcgctactaaaaattgttttgtatccttggtgggtgtcttctgctattttatctacttttgaacactttcaggactttttagccagtttgcctttcttgaaaaatgttatgttttcagcaataaatacatttgataatgaaaaaaaaaa

SEQ ID NO: 3 - protein, 15 aa, Artificial Sequence
  Description of Artificial Sequence Synthetic peptide
GGGGSGGGGSGGGGS